(19) United States
(12) Patent Application Publication
(10) Pub. No.: US 2015/0165187 A1
(43) Pub. Date: Jun. 18, 2015

(54) MAGNETIC APPARATUS FOR DIRECTING PERCUTANEOUS LEAD

(71) Applicant: Jung-Tung Liu, Taichung City (TW)

(72) Inventor: Jung-Tung Liu, Taichung City (TW)

(21) Appl. No.: 14/109,158

(22) Filed: Dec. 17, 2013

(51) Int. Cl. A61N 1/05

(52) U.S. Cl. CPC A61N 1/05; A61B 2017/00876

(57) ABSTRACT

A percutaneous lead apparatus is provided with a percutaneous lead including a first portion including at least one signal delivery electrode, a second portion including at least one connection terminal electrically connected to the at least one signal delivery electrode, a third portion having both ends coupled to the first portion and the second portion respectively, and a guide head disposed in a front end of the first portion wherein the percutaneous lead is a flexible, hollow, elongated tube; and a ferromagnetic member disposed in the guide head.

### BACKGROUND OF THE INVENTION

1. Field of the Invention

The invention relates to medical field and more particularly to a percutaneous lead apparatus having a magnetic arrangement for directing a percutaneous lead to a desired target tissue.

2. Description of Related Art

Neurological stimulators have been developed to relieve pain or the like. Implantable neurological stimulation systems generally have an implantable pulse generator and one or more electrode leads that deliver electrical pulses to neurological tissue or muscle tissue. There is a neurological stimulation system for spinal cord stimulation (SCS) has a plurality of cylindrical leads each including a lead body having a circular cross section and a plurality of spaced conductive rings (i.e., electrodes) at a distal end of the lead body. The SCS leads are implanted percutaneously through a large needle inserted into the epidural space. One concern of such leads is that the leads may not remain in the desired position after being implanted (i.e., migration). This is not desired because the stimulation provided by the electrodes may not be directed to the appropriate target tissue.

U.S. Pat. No. 7,146,222 addresses the potential for lead migration by providing structural reinforcement in at least one portion. However, this approach is principally directed to brain implants, and may not be effective for implantation at the spinal cord.

U.S. Pat. No. 8,108,052 discloses a lead that in turn includes first, second and third percutaneous portions. The first portion can carry an electrical contact, the second portion can be spaced apart from the first portion, and the third portion can be positioned between the first and second portions along a deployment axis. The lead includes one or more electrodes or electrical contacts that direct electrical signals into the patient's tissue to provide for patient relief.

U.S. Publication No. 20110071604 discloses a stimulation lead is configured to be implanted into a patient's body and includes at least one distal stimulation electrode and at least one conductive filer electrically coupled to the distal stimulation electrode. A jacket is provided for housing the conductive filer and providing a path distributed along at least a portion of the length of the lead for conducting induced RF energy from the filer to the patient's body. However, it is difficult of adjusting moving direction (e.g., forward, backward, left turn or right turn) of the lead when the lead is implanted percutaneously on the patient's body. As a result, the desired pain relief purpose is compromised.

Notwithstanding the prior art, the invention is neither taught nor rendered obvious thereby.

### SUMMARY OF THE INVENTION

It is therefore one object of the invention to provide a percutaneous lead including a first portion including at least one signal delivery electrode, a second portion including at least one connection terminal electrically connected to the at least one signal delivery electrode, a third portion having both ends coupled to the first portion and the second portion respectively, and a guide head disposed in a front end of the first portion wherein the percutaneous lead is a flexible, hollow, elongated tube; a ferromagnetic member disposed in the guide head; and a magnet configured to moveably place on a patient's body, thereby magnetically directing the guide head to a desired target tissue.

The above and other objects, features and advantages of the invention will become apparent from the following detailed description taken with the accompanying drawings.

### BRIEF DESCRIPTION OF THE DRAWINGS

FIG. 1 is a perspective view of a percutaneous lead apparatus according to a preferred embodiment of the invention;

FIG. 2 is a sectional view taken along line A-A of FIG. 1;

FIG. 3 is a schematic cross-sectional view showing an epidural needle inserted into an epidural space and a percutaneous lead inserted through the epidural needle into the epidural space;

FIG. 4 is a front cross-sectional view showing the percutaneous lead inserted into the epidural space and a magnet on the back of a patient;

FIG. 5 is a top cross-sectional view showing a medical employee watching a movement of the percutaneous lead in the dura mater of the spinal cord by using an x-ray detector, and a medical employee using a magnet to attract a ferromagnetic member so as to direct a movement of the percutaneous lead to slide on the dura mater toward a desired position in a patient's body; and

FIG. 6 is a view similar to FIG. 5 showing the percutaneous lead been disposed in a desired position in a patient's body.

### DETAILED DESCRIPTION OF THE INVENTION

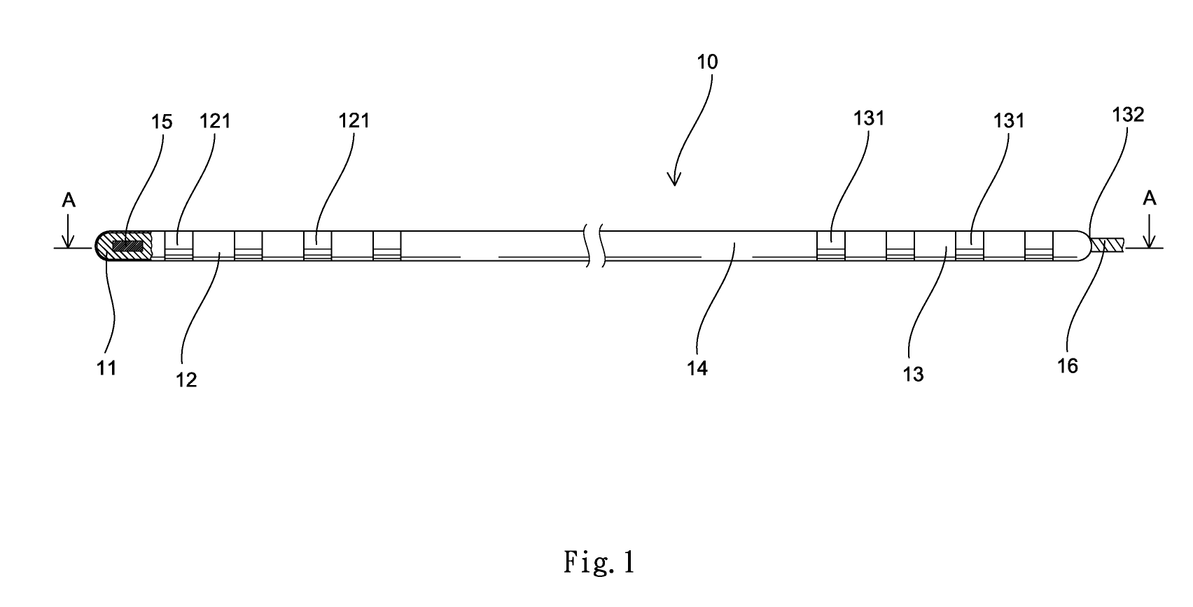

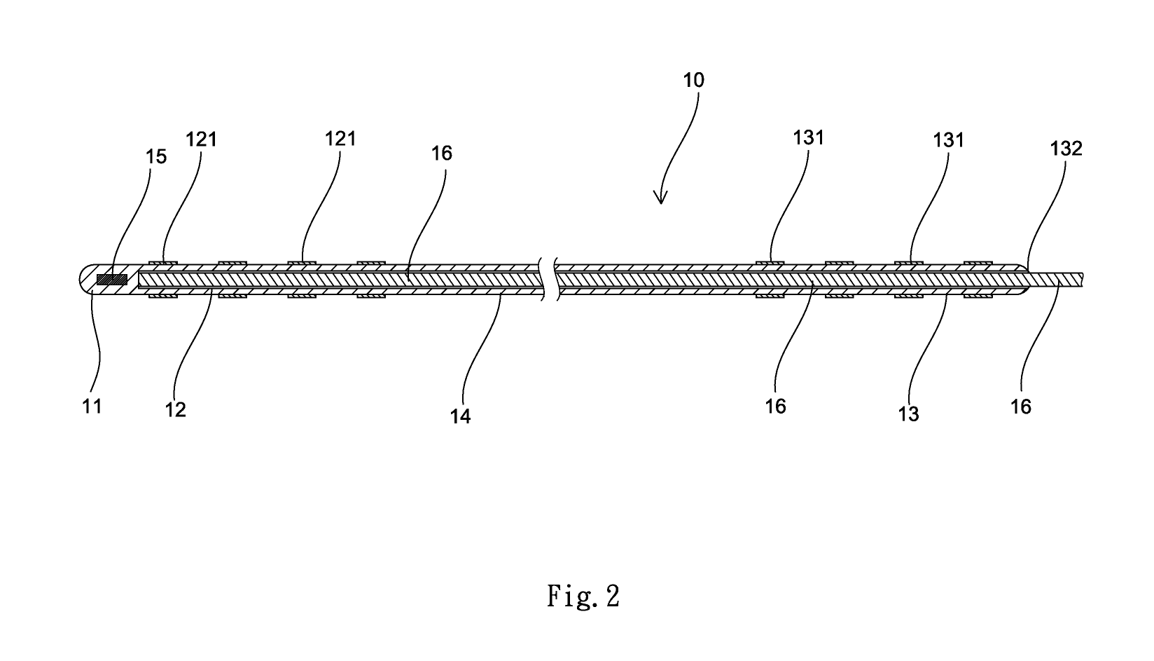

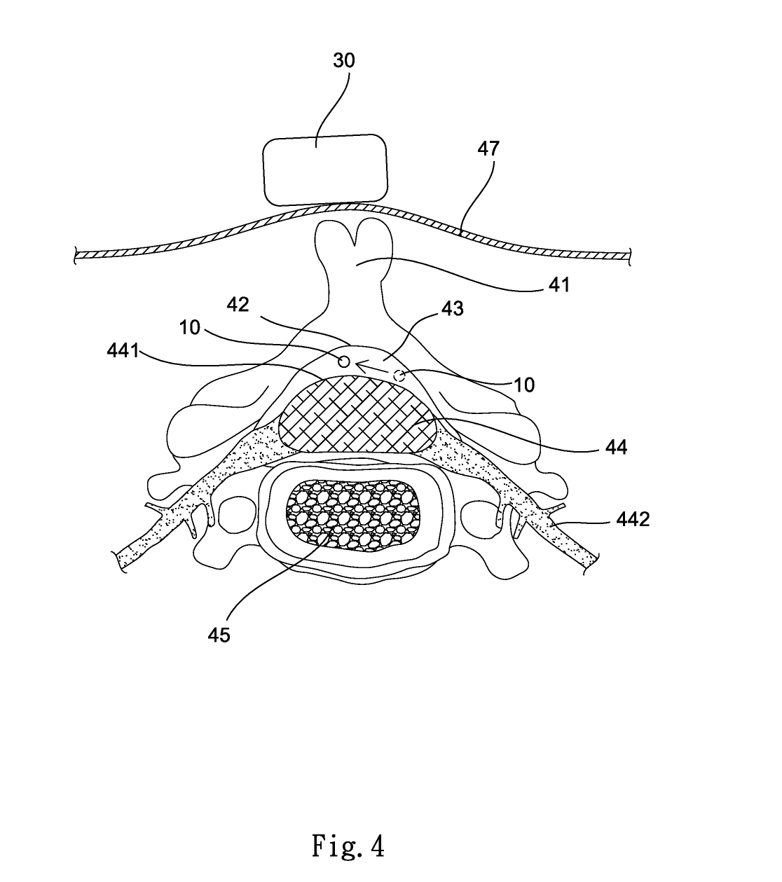

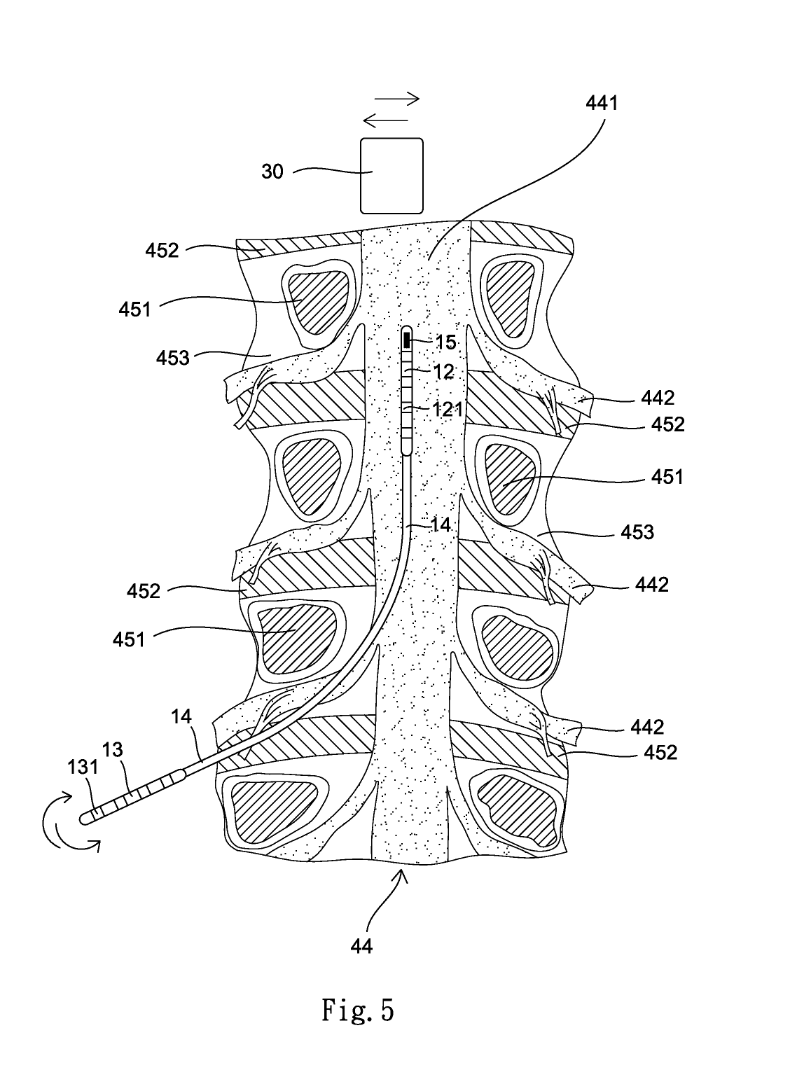

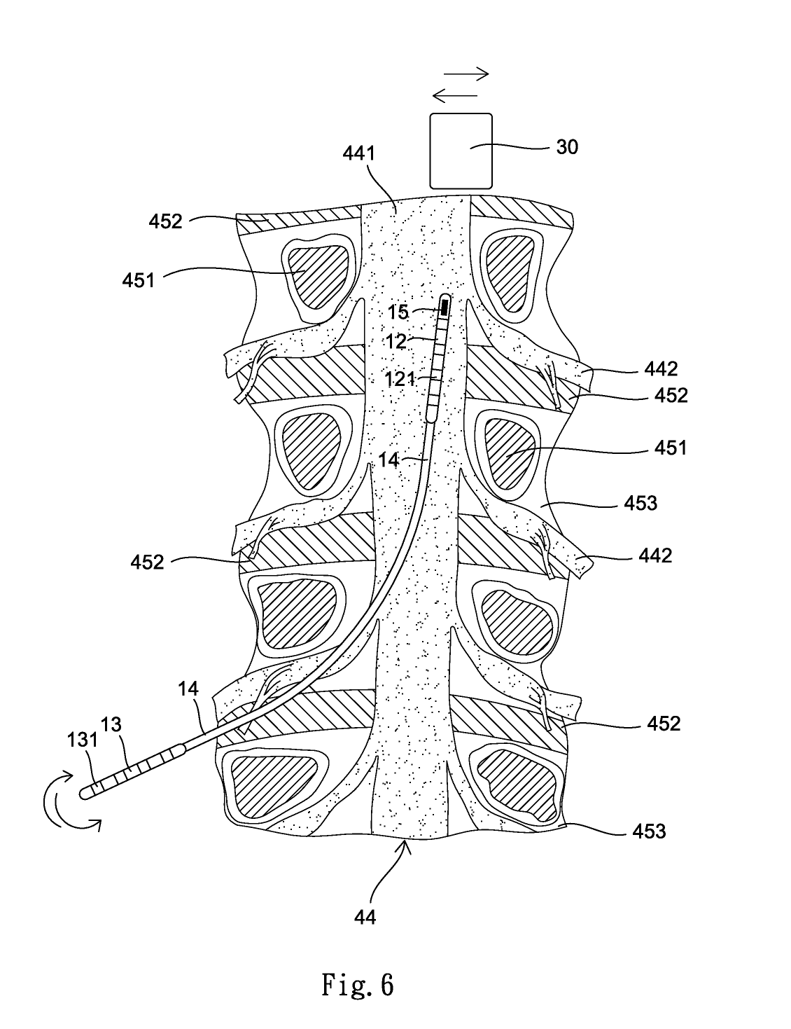

Referring to FIGS. 1 to 6, a percutaneous lead apparatus in accordance with a preferred embodiment of the invention comprises the following components as discussed in detail below.

A percutaneous lead 10 is a flexible, hollow, elongated tube and includes a guide head 11, a first portion 12, a second portion 13, and a third portion 14. The third portion 14 has both ends coupled to the first portion 12 and the second portion 13 respectively. The percutaneous lead 10 has a slender, elongated metal member (e.g., stylet) 16 as a delivery device which passes through the second, third, and first portions 13, 14, and 12 of the percutaneous lead 10 for increasing stiffness. Thus, the percutaneous lead 10 can be delivered percutaneously to the patient. Preferably, the metal member 16 is made of ferromagnetic material such as iron, nickel, cobalt, and most of their alloys. As a result, both the first portion 12 and the third portion 14 are ferromagnetic.

A ferromagnetic member 15 is disposed in the guide head 11 which is at a front end of the first portion 12. The first portion 12 comprises a plurality of spaced ring shaped signal delivery electrodes 121. The second portion 13 comprises a plurality of spaced ring shaped connection terminals 131 which are electrically connected to the signal delivery electrodes 121 and a pulse generator (not shown). The percutaneous lead 10 has an inner surface provided with multiple wires coupled between the signal delivery electrodes 121 and the corresponding terminals 131 so as to establish electrical links. A magnet 30 is moveably placed on the back 47 of a patient's body.

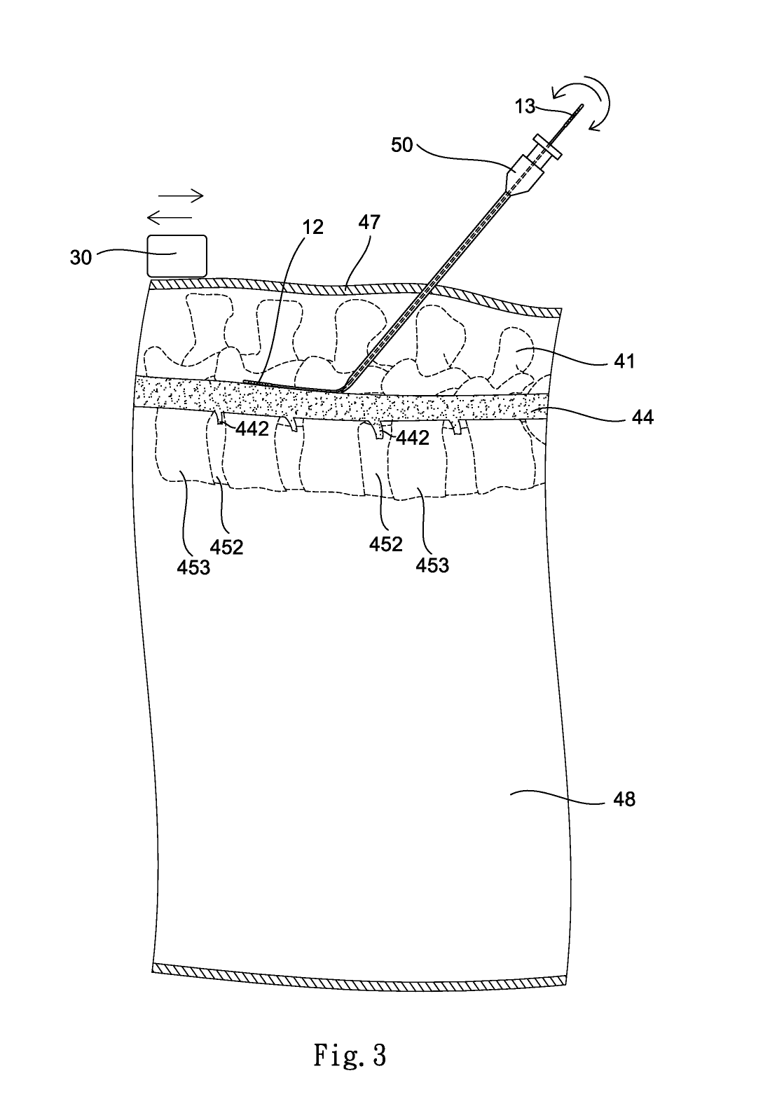

In operation (see FIGS. 3 to 6), a medical employee inserts an epidural needle 50 into a gap between two adjacent spinous processes 41 on the back 47 in which the needle head inserted into the epidural space 43 which is between a ligamentum flavum 42 and a dura mater 441 in a spinal cord 44. The spinal cord 44 has a plurality of spinal nerves 442. Next, the medical employee insert the percutaneous lead 10 into the epidural needle 50 until the front end of the signal delivery electrodes 121 hes the epidural space 43 with both the guide head 11 and the signal delivery electrodes 121 of the first portion 12 disposed in the epidural space 43, and the third portion 13 disposed externally of the patient's body. This facilitates a manual operation of the percutaneous lead 10. Thereafter, the medical employee may move the magnet 30 on the back 47 to attract the ferromagnetic member 15 in the guide head 11 and manipulate the third portion 13 so as to precisely move the guide head 11 toward the target tissue.

It is envisaged by the invention that the ferromagnetic member 15 (i.e., the percutaneous lead 10) can be precisely positioned by using an x-ray detector. Thus, the medical employee may move the magnet 30 on the back 47 to magnetically attract the ferromagnetic member 15 in order to direct the signal delivery electrodes 121 to an appropriate target tissue by using the x-ray detector. After reaching the target tissue, the medical employee may remove the slender, elongated metal member (e.g., the stylet) 16 out of a rear opening of the second portion 13. The connection terminals 131 of the second portion 13 are electrically connected to the signal delivery electrodes 121. Further, the connection terminals 131 of the second portion 13 are electrically connected to a port of the pulse generator (not shown). The pulse generator generates electrical signals and/or other types of signals to the connection terminals 131 and the signal delivery electrodes 121 which in turn deliver electrical pulses to neurological tissue (target tissue) in the epidural space 43 and onto the dura mater 441 for relieving pain.

Preferably, the angle of the epidural needle 50 with respect to the skin of the back 47 is 45-degree or less. As shown in FIG. 3-6, it shows the spinal cord 44 and the vertebra 45 between the human back 47 and the abdomen 48 in partial, cross-sectional views. The vertebra 45 includes pedicles 451, inervertebral discs 452, and vertebral bodies 453.

Preferably, the ferromagnetic member 15 is an alloy of metal, nickel, and cobalt.

While the invention has been described in terms of preferred embodiments, those skilled in the art will recognize that the invention can be practiced with modifications within the spirit and scope of the appended claims.

What is claimed is:

1. A percutaneous lead apparatus comprising:
a percutaneous lead comprising a first portion including at least one signal delivery electrode, a second portion including at least one connection terminal electrically connected to the at least one signal delivery electrode, a third portion having both ends coupled to the first portion and the second portion respectively, and a guide head disposed in a front end of the first portion wherein the percutaneous lead is a flexible, hollow, elongated tube; and
a ferromagnetic member disposed in the guide head.

2. The percutaneous lead apparatus of claim 1, further comprising a magnet configured to moveably place on a patient's body and configured to attract and move the ferromagnetic member.

3. The percutaneous lead apparatus of claim 1, wherein the ferromagnetic member is an alloy of metal, nickel, and cobalt.

4. The percutaneous lead apparatus of claim 1, further comprising a slender, elongated metal member as a delivery device which either passes through the second, third, and first portions when in use or is configured to remove out of a rear opening of the second portion when not in use.

5. The percutaneous lead apparatus of claim 4, wherein the slender, elongated metal member is of ferromagnetic material including iron, nickel, cobalt, and alloys thereof.

6. The percutaneous lead apparatus of claim 1, wherein the percutaneous lead has an inner surface provided with multiple wires coupled between the signal delivery electrodes and the corresponding terminals so as to establish electrical links.

7. The percutaneous lead apparatus of claim 1, wherein the at least one connection terminal of the second portion is electrically connected to a port of a pulse generator.

8. The percutaneous lead apparatus of claim 1, wherein the percutaneous lead is disposed through an epidural needle, wherein the epidural needle and the first portion are inserted through a patient's body, and the second portion is disposed externally of the patient's body.
